(19) United States
(12) Patent Application Publication
(10) Pub. No.: US 2023/0038953 A1
(43) Pub. Date: Feb. 9, 2023

(54) DEVICE FOR DISPENSING A FLUID PRODUCT

(71) Applicant: APTAR FRANCE SAS, Le Neubourg (FR)

(72) Inventor: Maxime HUPPÉ, Caudebec Les Elbeuf (FR)

(73) Assignee: APTAR FRANCE SAS, Le Neubourg (FR)

(21) Appl. No.: 17/787,087

(22) PCT Filed: Dec. 16, 2020
(86) PCT No.: PCT/FR2020/052461

§ 371 Date: Jun. 17, 2022

(30) Foreign Application Priority Data
Dec. 20, 2019 (FR) 1915092

(51) Int. Cl. A61M 5/142; G01F 11/02; A61M 5/315; A61M 5/168

(52) U.S. Cl. CPC A61M 5/1424; G01F 11/023; G01F 11/027; G01F 11/028; A61M 5/31515; A61M 5/31548; A61M 5/16881; A61M 2250/00

(57) ABSTRACT

Fluid dispensing device, having a pump connected to a reservoir and to a head. A piston (22) is connected to a piston rod (25) and provided with a push-button (26) to actuate the pump. A spring (27) brings the piston to the rest position after actuation. The body has a T-like connection (24), with a first branch connected to the metering chamber, a second branch connected to the reservoir and a third branch is connected to the head. A first valve (41) is provided in the second branch and a second valve (51) in the third branch, the valves are urged against the first and second respective valve seats (43, 53). The device has dose a adjustment (28, 29) cooperating with the piston rod and has a bush (29) coaxially around the piston rod movable to adjust the rest position of the piston.

The present invention relates to a device for dispensing a fluid product, in particular intended for the administration of a pharmaceutical product on small animals, such as for example, mice.

The devices of the state of the art used to administer doses of medications to small animals, such as rodents, in particular mice, are generally single-use, single-dose devices, in which all of the dose of product is dispensed in one single actuation. Yet, trials of medications on animals, in particular rodents, such as mice, are done generally on fifteen rodents, which therefore needs fifteen devices. In addition, the doses are generally not adjustable, such that a different device is needed for each different dosage.

U.S. Pat. No. 2,825,334, FR2343399 and U.S. Pat. No. 1,639,409 describe devices of the prior.

The purpose of this invention is to provide a fluid product dispensing device that does not have the above mentioned disadvantages.

In particular, an object of the present invention is to provide a fluid product dispensing device that allows to dispense several doses in several successive actuations.

Another object of the present invention is also to provide a fluid product dispensing device that makes it possible to adjust the dose before actuation.

Another object of the present invention is also to provide a fluid product dispensing device that is simple and reliable to use.

Another object of the present invention is also to provide a fluid product dispensing device that is simple and inexpensive to manufacture and to assemble.

The present invention therefore aims for a device or dispensing a fluid product comprising a pump connected, on the one hand, to a reservoir containing several doses of fluid product and, on the other hand, to a dispensing head provided with a dispensing opening, said pump comprising a body containing a metering chamber in which a piston connected to a piston rod provided with a push-button slides, on which the user presses to actuate the pump, a return spring cooperating with said piston and/or said piston rod to automatically return said piston to its rest position after each actuation, said body comprising downstream of said metering chamber, a T-like connection, of which a first branch is connected to said metering chamber, a second branch is connected to said reservoir and a third branch is connected to said dispensing head, a first valve being provided in said second branch of said T-like connection and a second valve being provided in said third branch of said T-like connection, said first and second valves being urged by the first and second springs against the first and second respective valve seats, said device comprising dose adjustment means cooperating with said piston rod, said dose adjustment means comprising a bush arranged coaxially around said piston rod, said bush being movable, in particular by screwing/unscrewing, with respect to said piston rod, to axially adjust the rest position of said piston in said body, and consequently the volume of said metering chamber.

Advantageously, said first and third branches are axially aligned, and said second branch extends radially with respect to said first and third branches.

Advantageously, said first and second valves are balls.

Advantageously, said first valve seat is formed in a first hollow sleeve fixed to said body of the pump and which defines said second branch of the T-like connection.

Advantageously, said second valve seat is formed in said body of the pump.

Advantageously, said reservoir is removably connected to said body.

Advantageously, said reservoir comprises a dip tube.

Advantageously, said dispensing head is removably connected to said body.

Advantageously, said dispensing head comprises an injecting needle provided with said dispensing opening.

Advantageously, said injecting needle is curved or bent.

In an advantageous variant, said dispensing head comprises a spray nozzle provided with said dispensing opening.

Advantageously, a hollow sleeve is inserted into said body, said metering chamber being defined inside said hollow sleeve.

Advantageously, a locking member, such as a screwable nut, is provided to lock the axial position of said bush on said piston rod.

These characteristics and advantages and others of the present invention appear more clearly from the following detailed description, given by way of non-limiting example, and with reference to the accompanying drawings, and in which:

FIG. 1 is a schematic cross-sectional view of a device for dispensing fluid product according to an advantageous embodiment, in position before use,

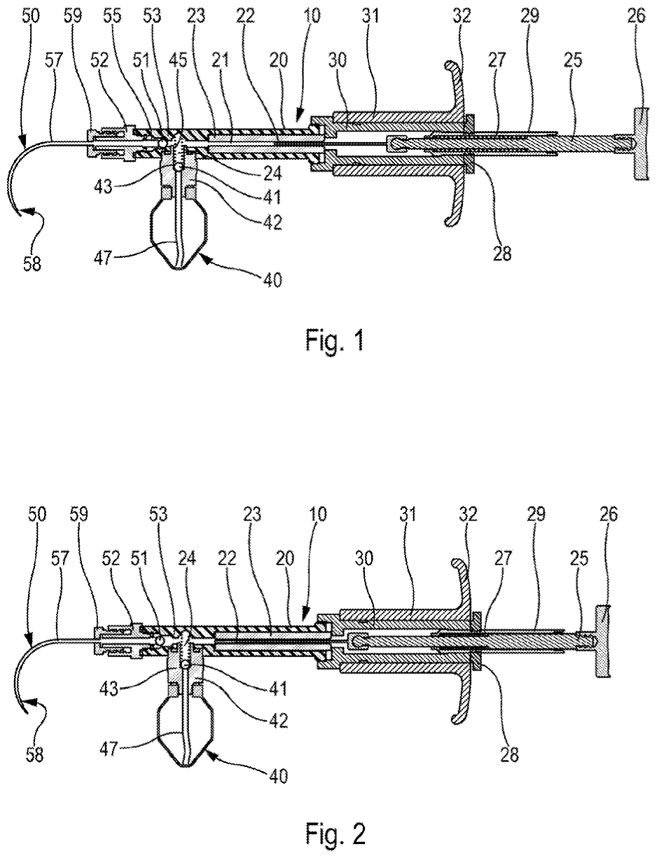

FIG. 2 is a view similar to that of FIG. 1, during initiation,

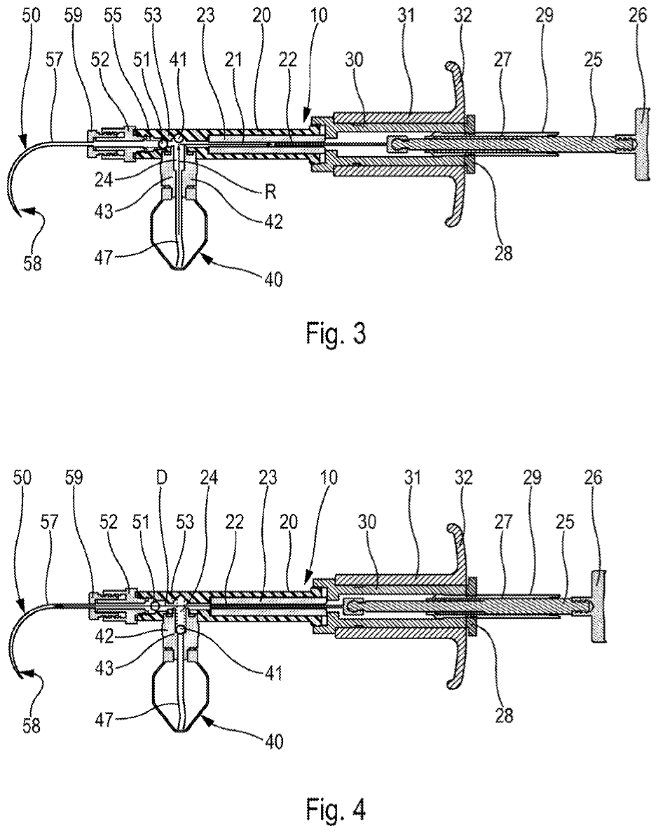

FIG. 3 is a view similar to that of FIG. 2, after initiation and before dispensing, and

FIG. 4 is a view similar to that of FIG. 3, during dispensing.

The terms “proximal” and “distal” are relative to the needle. The terms “upstream” and “downstream” are relative to the direction of flow of the fluid product during its dispensing. The terms “axial” and “radial” are relative to the longitudinal central axis of the device.

The device for dispensing a fluid product represented in the figures comprises a pump 10 connected, on the one hand, to a reservoir 40 containing several doses of fluid product and, on the other hand, to a dispensing head 50 provided with a dispensing opening 58.

The pump 10 comprises a body 20 containing a metering pump 21 in which a piston 22 slides.

In the example of the figures, a hollow sleeve 23 is inserted in the body 20, the metering chamber 21 being defined inside said hollow bush 23. Advantageously, this hollow sleeve 23 is easily interchangeable.

The piston 22 is connected to a piston rod 25 provided with a push-button 26 on which the user presses to actuate the pump 10. A return spring 27 cooperates with said piston 22 and/or said piston rod 25 to automatically return the piston to its rest position after each actuation, when the user stops pressing on said piston rod 25.

The body 20 is connected to a secondary body 30 surrounding at least partially the piston rod 25. An actuation element 31 can be provided around said secondary body 30, comprising a radial flange 32 on which the user presses to actuate the pump 10. In a variant, the radial flange 32 could be formed directly on said secondary body 30.

Dose adjustment means 28, 29 are provided between the secondary body 30 and the piston rod 25. These dose adjustment means comprise a bush 29 arranged coaxially around said piston rod 25, said bush 29 being movable, in particular by screwing/unscrewing, with respect to the piston rod 25, to axially adjust the rest position of the piston 22 in said body 20, and consequently the volume of the metering chamber 21.

A locking member 28, such as a screwable nut, is preferably provided to lock the axial position of said bush 29 on the piston rod 25.

Thus, to adjust the dose in the example represented in the drawings, it suffices to loosen the locking member 28, then to screw or unscrew the bush 29 with respect to the piston rod 25 to position the piston 22 in the desired position, and finally, retighten the locking member 28 to lock the bush 29.

Advantageously, adjustment indices can be provided, such as graduations, for example at the metering chamber 21. In this case, the body 20 can comprise a viewing window allowing to see the piston 22 in its rest position, or the body 20 could be made of a material at least partially transparent. Other adjustment indices are also possible, for example at the bush 29 and/or at the piston rod 25.

Using dose adjustment means allows in particular to change the dose according to the animal. For example, if tests relate to fifteen mice, the dose remains the same from one mouse to another. But, if the tests relate to fifteen mice and fifteen rats, then there is no need to change the device by changing the animal, thanks to the dose adjustment means.

The dose adjustment means also allow to adjust the dose according to the medication, for example if the tests relate to fifteen mice to be treated with a medication A dosed at 5 μL, and fifteen mice to be treated with a medication B dosed at 10 μL.

Typically, such dose adjustment means allow fine adjustments within a small dose, for example between 5 and 20 μL.

Using an interchangeable hollow sleeve 23 to define the metering chamber 21 also allows to modify the volume of the dose. Typically, this type of dose adjustment allows to go from a dose of 5 to 20 μL to a dose of 40 to 60 μL. Of course, these values are not limiting and are only given as an example.

The body 20 of the pump 10 comprises downstream of the metering chamber 21, a T-like connection 24, of which a first branch is connected to the metering chamber, a second branch is connected to the reservoir 40 and a third branch is connected to the dispensing head 50. In the example represented, the first and third branches are axially aligned, and the second branch extends radially with respect to said first and third branches.

A first valve 41 is provided in the proximity of the reservoir 40 in said second branch of the T-like connection 24. This first valve 41 can be formed by a ball urged by a first spring 45 against a first valve seat 43. Advantageously, this first valve seat 43 is formed in a first hollow sleeve 42 fixed to said body 20 of the pump 10 and which defines said second branch of the T-like connection 24. The first spring 45, in this case, bears in said body 20 to urge, at rest, the first valve 41 to its closing position.

A second valve 51 is provided in the proximity of the dispensing head 50, in said third branch of the T-like connection 24. This second valve 51 can be formed by a ball urged by a second spring 55 against a second valve seat 53. Advantageously, this second valve seat 53 is formed in said body 20 of the pump 10. The second spring 55, in this case, bears on a second hollow sleeve 52 fixed to the proximal axial end of said body 20 of the pump 10 to urge, at rest, the second valve 51 to its closing position.

The reservoir 40 is connected to the body 20, advantageously via said first hollow bush 42, and can comprise a dip tube 47. The fixing of the reservoir 40 on the first hollow bush 42 is advantageously removable to allow an easy replacement of an empty reservoir with a full reservoir.

The dispensing head 50 is connected to the body 20, advantageously via said second hollow sleeve 52, and can comprise an injecting needle 57 provided with the dispensing opening 58. In the example represented, the needle 57 is curved, but it could be straight or bent, or any suitable shape. It can be rigid, or in a variant, partially or totally flexible. It is provided with an end-fitting 59 to be fixed on the second hollow sleeve 52, in particular via a conventional fixing of the Luer Lock type. In a variant, it could be fixed directly on the body 20 of the pump 10.

According to another variant, the dispensing head 50 can comprise a spray nozzle incorporating the dispensing opening 58, to dispense the fluid product in spray form.

The operation of the device represented in the drawings will now be described in reference to the figures.

Before the first use, as can be seen in FIG. 1, the reservoir 40 contains all of the fluid product, the piston 22 is in the rest position under the effect of the return spring 27, and the first and second valves 41, 51 are in the closing position under the effect of the first and second springs 45, 55. The metering chamber 21 contains air.

When the user presses for the first time on the push-button 26, as can be seen in FIG. 2, it axially moves the piston rod 25 and therefore the piston 22 with respect to the body 20 and therefore to the metering chamber 21. The air contained in the metering chamber 21 and in the T-like connection 24 is compressed, by pushing the first valve 41 against its valve seat 43, and by pushing the second valve 51 away from its valve seat 53.

When the pressure exerted by the air exceeds the force of the second spring 55, the second valve 51 opens to allow the expulsion of the air and therefore the initiation of the pump.

When the user stops pressing on the push-button 26, as can be seen in FIG. 3, the return spring 27 returns the piston 22 and the piston rod 25 into the rest position. Likewise, the second spring 55 returns the second valve 51 into its closing position.

This axial return movement of the piston 22 generates a depression in the metering chamber 21 and in the T-like connection 24. This depression suctions the second valve 51 against its valve seat 53 and urges the first valve 41 away from its valve seat 43.

When this depression exceeds the force of the first spring 45, the first valve 41 opens and a dose of liquid is suctioned by depression into the metering chamber 21, via the T-like connection 24. This movement of the fluid is represented in FIG. 3 by the arrow R to symbolise the filling of the metering chamber 21. When the piston 22 is returned into the rest position, the first spring 45 again urges the first valve 41 to its closing position.

The device is thus ready for its first active use, to dispense a first dose of fluid product.

When the user presses for the second time on the push-button 26, as can be seen in FIG. 4, it axially moves the piston rod 25 and therefore the piston 22 with respect to the body 20 and therefore to the metering chamber 21. The fluid product contained in the metering chamber 21 and in the T-like connection 24 pushes the first valve 41 against its valve seat 43, and pushes the second valve 51 away from its valve seat 53.

When the pressure exerted by the fluid exceeds the force of the second spring 55, the second valve 51 opens to allow the expulsion of the fluid product to the dispensing head 50, and therefore the dispensing of the first dose of fluid product through the dispensing opening 58. This movement of the fluid is shown in FIG. 4 by the arrow D to symbolise the dispensing of the dose of fluid product.

When the user stops pressing on the push-button 26, the return spring 27 returns the piston 22 and the piston rod 25 into the rest position. Likewise, the second spring 55 returns the second valve 51 into its closing position. This axial return movement of the piston 22 generates a depression in the metering chamber 21 and in the T-like connection 24. This depression suctions the second valve 51 against its valve seat 53 and urges the first valve 41 away from its valve seat 43.

When this depression exceeds the force of the first spring 45, the first valve 41 opens and a dose of liquid is suctioned by depression into the metering chamber 21, via the T-like connection 24. When the piston 22 is returned into the rest position, the first spring 45 against urges the first valve 41 to its closing position.

The device is thus ready for its next active use, to dispense a next dose of fluid product.

Advantageously, when the reservoir 40 is empty, it can be easily replaced with a full reservoir and continue to use the same device. The position radially offset from the reservoir 40 allows to perform this operation of substituting reservoirs very easily.

Although the present invention is described above with reference to an advantageous embodiment, naturally various modifications can be applied thereto by the person skilled in the art, without going beyond the scope of the present invention, as defined by the accompanying claims.

1. Device for dispensing a fluid product comprising a pump (10) connected, on the one hand, to a reservoir (40) containing several doses of fluid product and, on the other hand, to a dispensing head (50) provided with a dispensing opening (58), said pump (10) comprising a body (20) containing a metering chamber (21) in which a piston (22) connected to a piston rod (25) provided with a push-button (26) slides, on which the user presses to actuate the pump (10), a return spring (27) cooperating with said piston (22) and/or said piston rod (25) to automatically return said piston (22) to its rest position after each actuation, characterised in that said body (20) comprises downstream of said metering chamber (21), a T-like connection (24), of which a first branch is connected to said metering chamber (21), a second branch is connected to said reservoir (40) and a third branch is connected to said dispensing head (50), a first valve (41) being provided in said second branch of said T-like connection (24) and a second valve (51) being provided in said third branch of said T-like connection (24), said first and second valves (41, 51) being urged by first and second springs (45, 55) against first and second respective valve seats (43, 53), said device comprising dose adjustment means (28, 29) cooperating with said piston rod (25), said dose adjustment means comprising a bush (29) arranged coaxially around said piston rod (25), said bush (29) being movable, in particular by screwing/unscrewing, with respect to said piston rod (25), to axially adjust the rest position of said piston (22) in said body (20), and consequently the volume of said metering chamber (21).

2. Device according to claim 1, wherein said first and third branches are axially aligned, and said second branch extends radially with respect to said first and third branches.

3. Device according to claim 1, wherein said first and second valves (41, 51) are balls.

4. Device according to claim 1, wherein said first valve seat (43) is formed in a first hollow bush (42) fixed to said body (20) of the pump (10) and which defines said second branch of the T-like connection (24).

5. Device according to claim 1, wherein said second valve seat (53) is formed in said body (20) of the pump (10).

6. Device according to claim 1, wherein said reservoir (40) is removably connected to said body (20).

7. Device according to claim 1, wherein said reservoir (40) comprises a dip tube (47).

8. Device according to claim 1, wherein said dispensing head (50) is removably connected to said body (20).

9. Device according to claim 1, wherein said dispensing head (50) comprises an injecting needle (57) provided with said dispensing opening (58).

10. Device according to claim 9, wherein said injecting needle (57) is curved or bent.

11. Device according to claim 1, wherein said dispensing head (50) comprises a spray nozzle of said dispensing opening (58).

12. Device according to claim 1, wherein a hollow sleeve (23) is inserted into said body (20), said metering chamber (21) being defined inside said hollow sleeve (23).

13. Device according to claim 1, wherein a locking member (28) such as a screwable nut, is provided to lock the axial position of said bush (29) on said piston rod (25).
